(19) United States
(12) Patent Application Publication
(10) Pub. No.: US 2005/0288546 A1
(43) Pub. Date: Dec. 29, 2005

(54) Device employing camera connector

(76) Inventors: Elazar Sonnenschein, Beer-Sheva (IL); Yuval Elovici, Shtulim (IL); Gary L. Long, Cincinnati, OH (US); Gregory J. Bakos, Mason, OH (US); Christopher Paul Swain, London (GB)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/127,514

(22) Filed: May 12, 2005

Related U.S. Application Data
(60) Provisional application No. 60/571,116, filed on May 14, 2004.
(60) Provisional application No. 60/571,118, filed on May 14, 2004.

(51) Int. Cl. A61B 1/00

(52) U.S. Cl. 600/101

(57) ABSTRACT

An endoscopic system is disclosed, and can include a disposable catheter, a reusable camera assembly, and a camera connector system. A small cable and cable connector for feeding through a narrow channel of the disposable catheter is disclosed. A connector for connecting to a video console via an improved connector assembly system is also disclosed.

### CROSS-REFERENCES TO RELATED APPLICATIONS

This application claims priority to and incorporates by reference U.S. Provisional Patent Application 60/571,116 “Medical Device Employing Endoscope System with a Disposable Catheter, a Reusable Camera Assembly, and an Improved Camera Connector System”, filed May 14, 2004; and U.S. Provisional Patent Application 60/571,118, “Medical Device with Propulsion Mechanism” filed May 14, 2004.

### FIELD OF THE INVENTION

This invention relates to endoscopic devices and methods in general, and more particularly to endoscopic devices and methods which may employ a disposable catheter and a reusable camera removable via a connector system.

### BACKGROUND

In recent years, the endoscope has been used widely in both the medical art and industrial art fields. For example, an endoscope which is used in the medical art field has an inserting section (i.e., a catheter) that contains a vision system, which is inserted into a body cavity, so as to be able to observe internal organs, and which may also be used as a conduit for a treatment instrument or tool inserted into a treating-instrument channel (or working channel) in the endoscope, so as to be able to perform various types of medical care or treatment. Additionally, a typical endoscope includes an articulation mechanism for controlling the motion of the catheter during an endoscope inspection procedure. Consequently, an endoscope that includes, for example, a vision system, an articulation mechanism, and other electronic or mechanical devices, can be relatively expensive. Because of its high cost, it can be desirable that the endoscope device be reusable. Therefore, it must be able to withstand a cleaning and disinfection process. This requirement adds even further complexity, thus cost, to the endoscope.

Generally, in a case in which an endoscope which has been used once for inspection and treatment within a body cavity is again used for other patients, disinfection chemicals, or an ethylene oxide gas, are used to perform washing or cleaning and disinfection of the endoscope after completion of the inspection and the treatment, in order to prevent the transmission of infection from patient to patient by means of the endoscope. However, as about one hour may be required to sufficiently disinfect the endoscope, the cleaning and disinfection time becomes a dead time. In a five-hour time span, for example, if it is assumed that the time per each procedure is fifteen minutes, only four persons can be inspected by a single endoscope. Consequently, a physician's capacity to treat several patients in a day is limited and thereby creates a backlog of patients waiting for this procedure, which creates a further problem, in that patients are not being treated in a timely manner.

In order to reduce the time required, multiple endoscopes may be made available for use. More specifically, if a plurality of endoscopes are prepared, and if the endoscopes which have been cleaned and disinfected are used during the cleaning and disinfection time of other endoscopes, the number of inspection procedures performed in a given time frame is increased. In this case, however, there occurs a problem, in that the purchase of multiple endoscopes is required, which represents a capital investment by the physician.

As a result, a physician's daily capacity can be limited by the number of endoscope devices he/she has available, which is further limited by the number of devices a doctor can afford to purchase, given their expense. Therefore, the overall number of procedures performed per day is limited by (1) the amount of time it takes to clean the endoscope device after each use and (2) the total number of endoscope devices a doctor can afford to purchase. Consequently, what is needed is a way to increase a physician's daily throughput in relation to the number of endoscope inspection procedures performed in a day. Further, what is needed is a way to cost-effectively increase this daily throughput.

U.S. Pat. No. 5,682,199, entitled, “Video endoscope with interchangeable endoscope heads.” Discloses a video endoscope with interchangeable endoscope heads. While the '199 patent describes a suitable endoscope for performing inspection procedures, scientists and engineers continue to seek ways of increasing a physician's daily throughput in relation to the number of endoscope inspection procedures performed in a day, while providing cost-effective inspection procedures.

Vision systems for use with endoscopes may include, for example, a charged coupled device (CCD) camera arranged at a forward end of an inserting section of the endoscope for projecting a color endoscope image on a screen of a video monitor unit. A CCD is a light-sensitive device that is used to turn the light entering though the lens into electronic signals that can be digitally processed and saved.

### SUMMARY OF THE INVENTION

Applicants have recognized the desirability of an endoscope system with a technically practical and cost-effective combination of both disposable and non-disposable components, such as a CCD camera, and which can provide a cost-effective method of increasing a physician's daily throughput in relation to the number of endoscope inspection procedures performed.

Applicant's have also recognized the desirability of providing a relatively small (i.e., less than about 2.8 mm width), easy-to-operate connector system that has multiple electrical connections for insertion in a small channel of medical devices, such as an endoscope, which connector can employ multiple electrical connections and that is easily scalable to more electrical connections, and which suitably small connector is cleanable with standard cleaning substances, such as a Cidex Solution.

In one embodiment, the invention provides a medical device useful in endoscopic procedures. The device comprises an elongate flexible member adapted for extending from a point outside a patient's body to a point inside the patient's body, and having at least one passageway communicating from a point outside the body to a point inside the body. The passageway has a distal end and a proximal end. The medical device also includes a camera adapted to be disposed at a distal portion of the elongated flexible member within the body for receiving images within the patient's body. The medical device also includes an assembly operatively associated with the camera and sized to be receivable in the distal end of the passageway and to pass through the proximal end of the passageway in the elongated flexible member. The assembly is positioned outside of the patient's body during use of the medical device. The assembly can be a camera cable end assembly. The cable camera end assembly can comprise circuitry, such as in the form of a printed circuit board, which is sized to pass through the passageway in the elongate flexible member.

In one embodiment, the present invention provides an endoscope system with a flexible, elongated disposable catheter, a reusable camera assembly, and an improved camera connector system. The endoscope system can include a reusable camera assembly that has a suitably small cable and cable connector for feeding through a narrow channel of the disposable catheter and, subsequently, connecting to a video console via a novel connector assembly system. As a result, the endoscope system of the present invention provides a cost-effective method of increasing a physician's daily throughput in relation to the number of endoscope inspection procedures performed by allowing several reusable camera assemblies and disposable catheters to be purchased and, thus, be available for use.

Accordingly, instead of requiring that a physician or hospital keep a certain number of reusable endoscope systems on hand and cycling those through a cleaning process, as is customary, a greater number of less expensive reusable camera assemblies can be provided and cycled through the cleaning process with each use, thereby increasing a physician's daily throughput in relation to the number of endoscope inspection procedures performed.

### BRIEF DESCRIPTION OF THE DRAWINGS

While the novel features of the invention are set forth with particularity in the appended claims, the invention in all its embodiments may be more fully understood with reference to the following description and accompanying drawings.

FIG. 1 illustrates an endoscope system that includes a disposable catheter and a removable camera in accordance with the invention.

FIG. 2 illustrates a perspective view of a catheter distal end of the disposable catheter in accordance with the invention.

FIG. 3 illustrates an exploded perspective view of a connector assembly in combination with a camera cable and a ferrite assembly in accordance with the invention.

FIG. 4 illustrates a perspective view of a lower housing in accordance with the invention.

FIG. 5 illustrates a perspective view of an upper housing in accordance with the invention.

FIG. 6 illustrates a perspective view of a frame in accordance with the invention.

FIG. 7 illustrates a top view of a camera PCB in accordance with the invention.

FIG. 8 illustrates a top view of a console PCB in accordance with the invention.

FIG. 9 illustrates an electrical schematic diagram of the endoscope system that relates to the signal flow from the video console to the removable camera in accordance with the invention.

FIGS. 10A and 10B illustrate a cross-sectional view along the length of the ferrite assembly and an end view of the ferrite assembly, respectively, in accordance with the invention.

FIG. 11 illustrates example dimensions of a first adhesive gasket used in the connector assembly of the present invention.

FIG. 12 illustrates example dimensions of a second adhesive gasket used in the connector assembly of the present invention.

FIG. 13 illustrates a flow diagram of a method of assembling the connector assembly to a video console in accordance with the invention.

FIG. 14 illustrates a flow diagram of a method of using the endoscope system of the present invention.

FIG. 15 illustrates a perspective view of the endoscope system positioned relative to a body cavity.

### DETAILED DESCRIPTION OF THE INVENTION

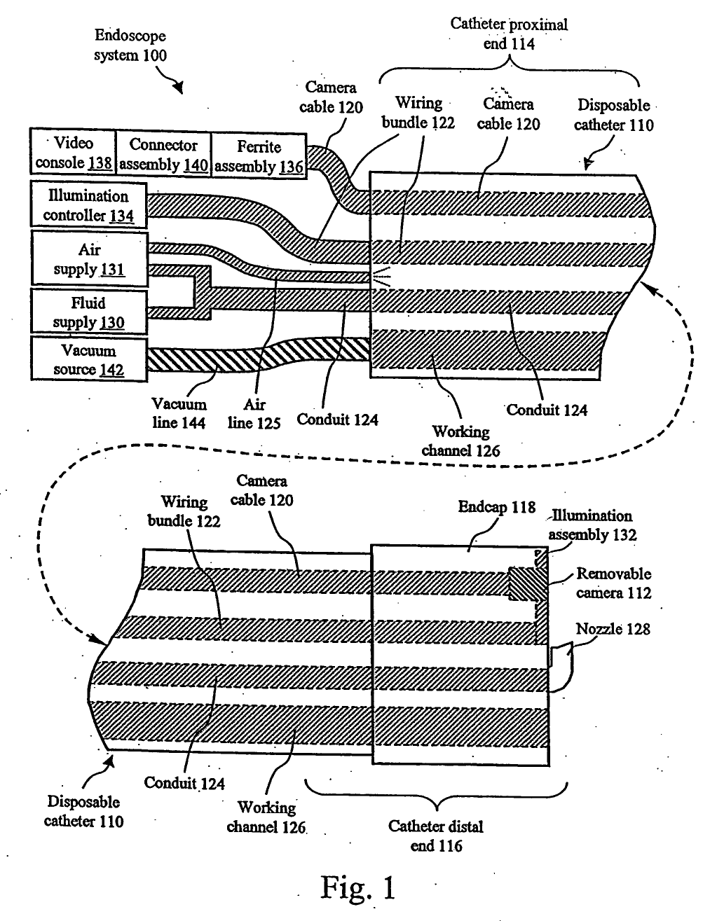

FIG. 1 illustrates an endoscope system 100 that includes a disposable catheter 110 and a removable camera 112 in accordance with one embodiment of the present invention. Disposable catheter 110 of endoscope system 100 has a catheter proximal end 114 for connecting to various peripheral devices and a catheter distal end 116 with an endcap 118 for insertion into a body cavity of a patient for performing medical inspections. Disposable catheter 110 is flexible tubing formed of, for example, polyurethane or FEP (Teflon derivative) tube, within which are several channels along its full length for installing a camera cable 120, a wiring bundle 122, a conduit 124, and a working channel 126 for inserting various instruments as needed during an inspection procedure.

Removable camera 112 is any commercially available visualization device that is available in sizes adaptable for use in disposable catheter 110, for example, a commercially available CCD camera, such as Medigus Visum 110, or a complementary metal-oxide semiconductor (CMOS) camera. A suitable CMOS camera uses a CMOS image sensor, such as model number OV7620 from OmniVision Technologies (Sunnyvale, Calif.).

Conduit 124 may be permanently affixed within disposable catheter 110. More specifically, a first end of conduit 124 may be connected to a nozzle 128 that is installed in and may protrude from endcap 118 at catheter distal end 116. Nozzle 128 is an outlet that is angled at 90° toward the lens of removable camera 112, which is also installed within endcap 118 at catheter distal end 116. Nozzle 128 is formed of, for example, stainless steel 316. A second end of conduit 124 extends out of disposable catheter 110 at catheter proximal end 114 and is hydraulically connected to a fluid supply 130 and an air supply 131. Fluid supply 130 is the source of a cleaning fluid, such as water, that passes through conduit 124 under pressure (by pumping) and subsequently exits nozzle 128 and flushes across a transparent protective cap (not shown) that is above the lens of removable camera 112 and through which nozzle 128 protrudes, thereby cleaning removable camera 112 as needed. Air supply 131 provides a source of air flow that may also pass through conduit 124 under pressure (by pumping) and subsequently exits nozzle 128 for providing insufflation gas (such as for instance carbon dioxide or air) to provide the distention of a body cavity for the purpose of effective visualization. In operation, the user may select either fluid from fluid supply 130, or alternatively, air from air supply 131 to be discharged from nozzle 128 at catheter distal end 116 of disposable catheter 110. In the case of this device and in other endoscopes, the airflow may be used to pressurize a resevoir containing fluid, so when the fluid valve is opened, it is “pushed” by the air, but doesn't mix with it unless the resevoir becomes empty.

Air can be pumped from air supply 131 via an air line 125 into the empty space surrounding the collective group of camera cable 120, wiring bundle 122, conduit 124, and air line 125 within disposable catheter 110. The airflow circulated through disposable catheter 110 serves to cool all elements within disposable catheter 110. In particular, this airflow may be used for cooling the active elements of an illumination assembly 132 that is installed within endcap 118 at catheter distal end 116 of disposable catheter 110.

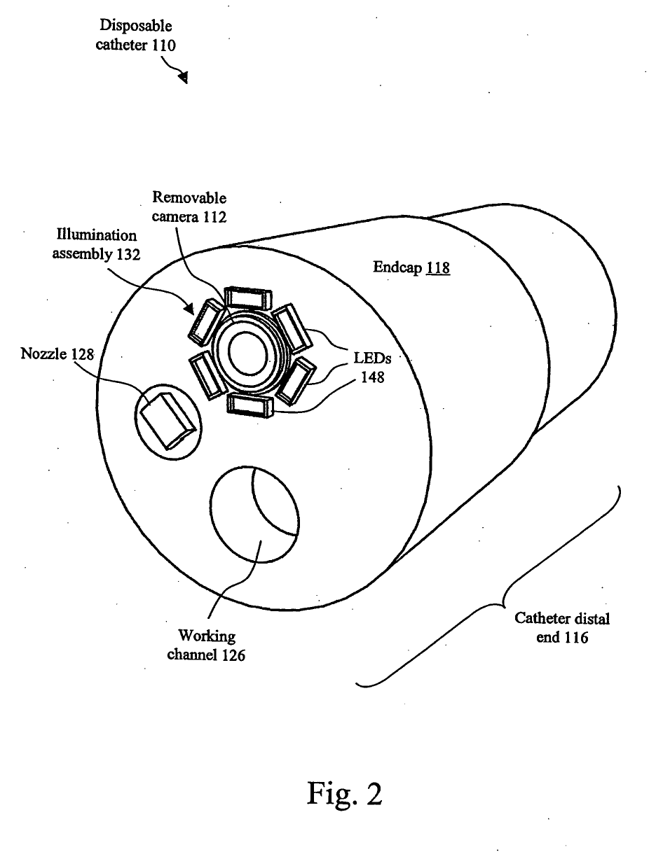

Illumination assembly 132 provides light to the tissue of the inspection area. Illumination assembly 132 is electrically connected to a first end of wiring bundle 122, which is installed along the length of disposable catheter 110. A second end of wiring bundle 122 extends out of disposable catheter 110 at catheter proximal end 114 and is electrically connected to an illumination controller 134, which provides the on/off control of illumination assembly 132. Wiring bundle 122 is representative of multiple electrical conductors for transmitting signals and power from illumination controller 134 to illumination assembly 132. FIG. 2 describes further details of illumination assembly 132 in combination with removable camera 112.

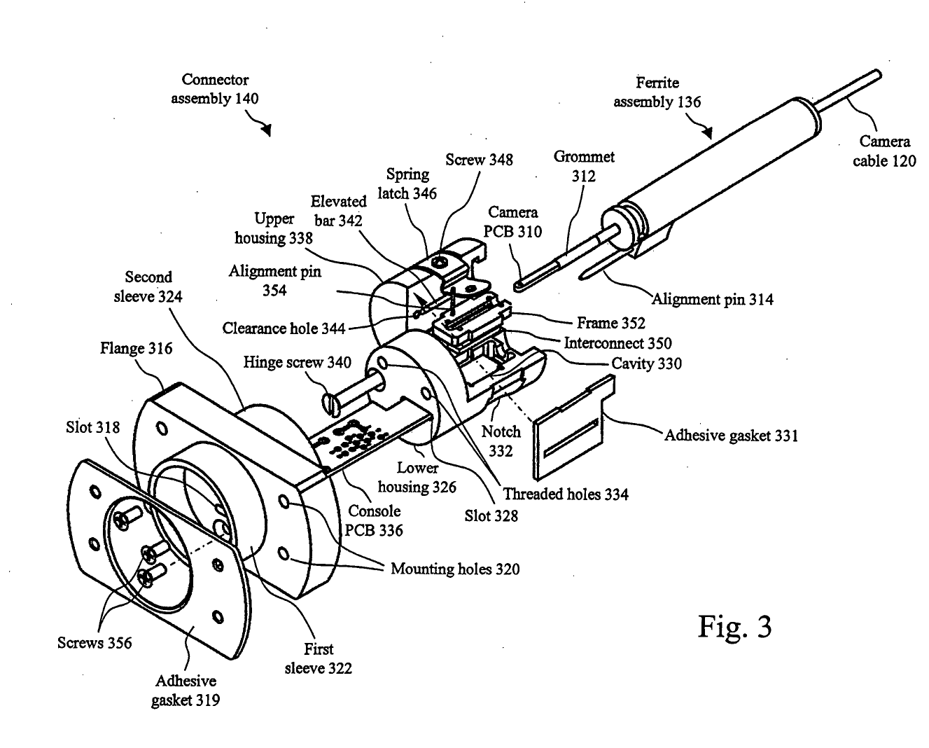

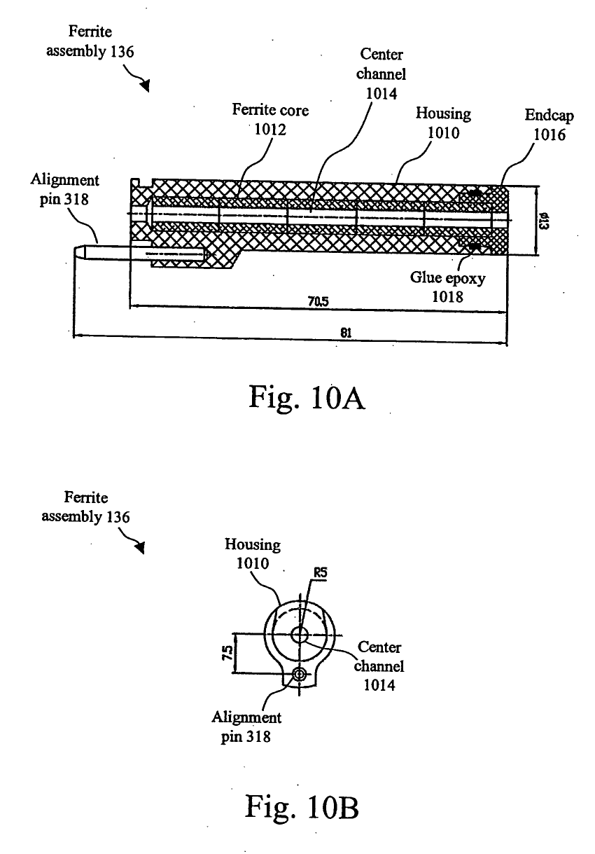

Removable camera 112 that is installed within endcap 118 at catheter distal end 116 is electrically connected to a first end of camera cable 120, which is installed along the length of disposable catheter 110. A second end of camera cable 120 extends out of disposable catheter 110 at catheter proximal end 114, passes through a ferrite block 136, and is electrically connected to a video console 138 via a connector assembly 140. Camera cable 120 is representative of multiple electrical conductors used for transmitting signals and power from video console 138 to removable camera 112. FIG. 3 describes further details of camera cable 120, ferrite block 136, and connector assembly 140. FIGS. 10A and 10B also describe further details of ferrite block 136.

As an example use for working channel 126, FIG. 1 shows a vacuum source 142 connected to a vacuum line 144 that is fed into working channel 126 to catheter distal end 116 for providing suction to the inspection area when endoscope system 100 is in use.

Generally, working channel 126 allows the insertion of medical instruments, such as balloons, dilators, tissue graspers, tissue cutting devices, tissue stapling devices, tissue staining or treatment devices, vessel ligation devices, biopsy forceps, and tissue ablation devices, through disposable catheter 110, thereby accessing a desired tissue site to assist in diagnosis and treatment of tissue.

Endoscope system 100 of the present invention is not limited to the configuration shown in FIG. 1. Disposable catheter 110 of endoscope system 100 may include other channels, such as other fluid (gas or liquid) channels may extend through disposable catheter 110 to supply, for example, water, saline solution, pharmaceuticals, or lubricating fluids.

FIG. 2 illustrates a perspective view of catheter distal end 116 of disposable catheter 110 in accordance with one embodiment of the invention. FIG. 2 shows the arrangement of removable camera 112, working channel 126, nozzle 128 and illumination assembly 132 installed within endcap 118. Illumination assembly 132 may be formed by a plurality of light emitting diodes (LEDs) 148 that are mounted upon an associated printed circuit board (PCB) (not shown) and arranged around the perimeter of removable camera 112. Furthermore, the PCB of illumination assembly 132 has a clearance hole (not shown) that allows removable camera 112 to be inserted therethrough. The PCB of illumination assembly 132 is electrically connected to wiring bundle 122, which is connected to illumination controller 134. When initiated by the user, illumination controller 134 provides the electrical stimulus to activate LEDs 148, thereby lighting the tissue of the inspection area. The transparent protective cap (not shown) at the tip of catheter distal end 116 isolates the human tissue from the LEDs 148 when endoscope system 100 is in use.

With reference to FIGS. 1 and 2, the general initial assembly of endoscope system 100 may be performed as follows. Disposable catheter 110 is manufactured having working channel 126, conduit 124, and wiring bundle 122 installed therein, but not yet connected to, fluid supply 130 and illumination controller 134. Initially, endcap 118 is not installed upon catheter distal end 116 of disposable catheter 110; instead, endcap 118 is hanging separately from disposable catheter 110 by wiring bundle 122, such that the channel within disposable catheter 110 for accepting camera cable 120 is accessible. Removable camera 112 and camera cable 120 are preassembled. The proximal end of camera cable 120 is then inserted into catheter distal end 116 of disposable catheter 110 and pushed along the full length of disposable catheter 110 until camera cable 120 emerges from catheter proximal end 114 of disposable catheter 110, is threaded through ferrite block 136, and connected to video console 138 via connector assembly 140, as described in more detail in reference to FIG. 3. Removable camera 112 is subsequently installed into catheter distal end 116 of disposable catheter 110. More specifically, removable camera 112 snaps into a square, recessed hole (not shown) that has a rubber gasket (not shown) to hold removable camera 112 in place by pressure. Once removable camera 112 is installed, endcap 118 is secured upon catheter distal end 116 of disposable catheter 110, such that removable camera 112 is aligned with illumination assembly 132, as shown in FIG. 2. Fluid supply 124 extends straight from catheter distal end 116 and through endcap 118 and serves as an alignment aid when assembling endcap 118 onto catheter distal end 116. Conduit 124 and wiring bundle 122 are then coupled to fluid supply 130 and illumination controller 134, respectively; thus, endoscope system 100 is ready for use.

After each use of endoscope system 100, camera cable 120, conduit 124, and wiring bundle 122 can be disconnected from video console 138, fluid supply 130, and illumination controller 134, respectively. Endcap 118 is disassembled from catheter distal end 116 of disposable catheter 110, thereby exposing removable camera 112. Removable camera 112 is removed from disposable catheter 110 by pulling its camera cable 120 out of disposable catheter 110. Disposable catheter 110 can then be discarded, and the assembly of removable camera 112 and camera cable 120 are cleaned and disinfected by soaking in, for example, a standard cleaning substance, such as a Cidex Solution supplied by Johnson & Johnson (New Brunswick, N.J.).

The removability and reuse of removable camera 112 from disposable catheter 110 is enabled by an electrical connector installed upon the proximal end of camera cable 120 that is suitably small enough to be threaded through a small channel (i.e., less than 2.8 mm diameter), within disposable catheter 110. Connector assembly 140 provides a suitable means for easily and quickly attaching or detaching camera cable 120 to/from video console 138, as described in more detail in reference to FIGS. 3, 4, 5, 6, 7, 8, 9, 10A, and 10B.

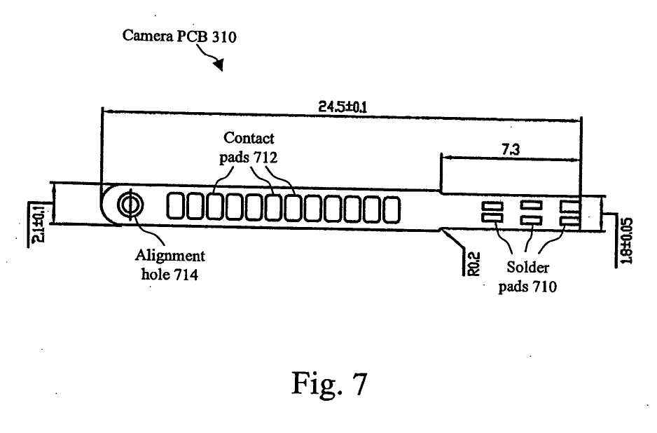

FIG. 3 illustrates an exploded perspective view of connector assembly 140 in combination with camera cable 120 and ferrite assembly 136 in accordance with the invention. FIG. 3 shows a camera PCB 310 that is electrically and mechanically connected at the proximal end of camera cable 120. Camera PCB 310 is a standard, multilayer PCB. Camera PCB 310 is described in more detail in reference to FIG. 7. A standard grommet 312 is installed at the electrical interface between camera PCB 310 and camera cable 120 and is used for providing a protective covering for handling. As is well known, grommet 312 provides a place to grip the camera cable 120-side of camera PCB 310. Grommet 312 is, for example, 18 mm in length, has an outside diameter of 2.4 mm, and has an inside diameter of 2.2 mm. The combination of camera cable 120, camera PCB 310, and grommet 312 are inserted through a center channel along the length of ferrite assembly 136. Ferrite assembly 136 is described in more detail in reference to FIGS. 10A and 10B. As shown in FIG. 3, however, ferrite assembly 136 includes an alignment pin 314 installed on the connector assembly 140 side of ferrite assembly 136.

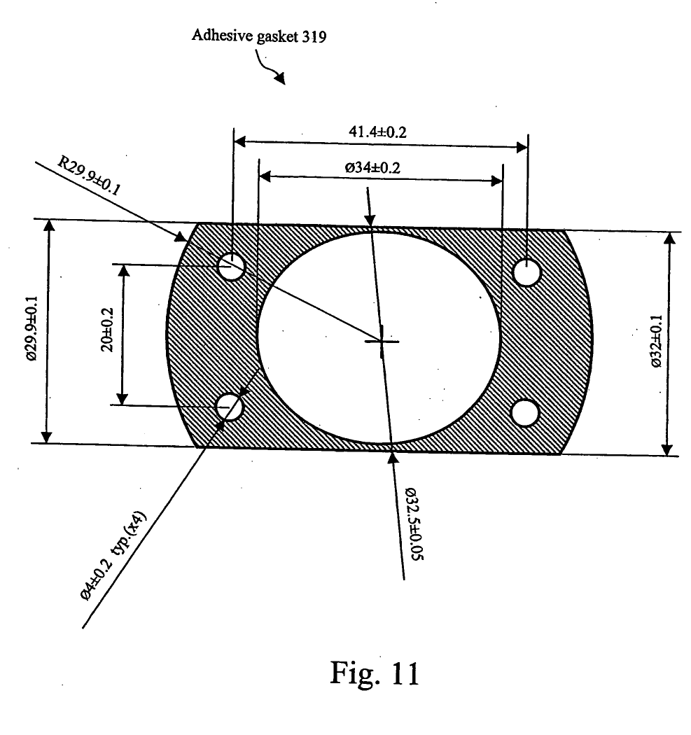

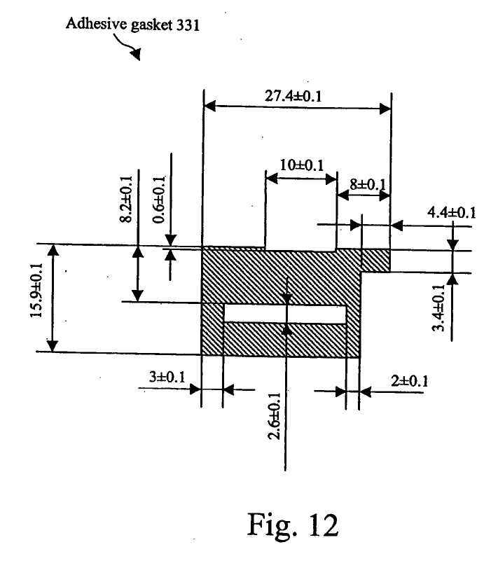

Connector assembly 140 includes a flange 316 that has a slot 318, multiple mounting holes 320, a first sleeve 322, and a second sleeve 324 mounted on opposing sides; a lower housing 326 that has a slot 328, a cavity 330, a notch 332, and multiple threaded holes 334; a console PCB 336 fitted within slot 318 of flange 316 and slot 328 of lower housing 326; an upper housing 338 hingeably connected to lower housing 326 via a hinge screw 340; an elevated bar 342 and a clearance hole 344 within upper housing 338; a spring latch 346 mounted to upper housing 338 via a screw 348; an interconnect 350 sandwiched between a frame 352 and an upper surface of console PCB 336, an alignment pin 354, multiple screws 356; a first adhesive gasket 319 that is applied to the video console 138-side of flange 316; and a second adhesive gasket 331 that is applied to the underside of upper housing 338. Adhesive gasket 319 and adhesive gasket 331 are described in more detail in reference to FIGS. 11 and 12, respectively.

Flange 316 can be formed of molded plastic or other non-conductive material. First sleeve 322 of flange 316 is mounted inside video console 138 via a hole in its front panel. Flange 316 is mounted to the front panel of video console 138 via four screws (not shown) that are installed at mounting holes 320. A first end of console PCB 336 passes through slot 318, thereby entering the front panel of video console 138. Consequently, slot 318 is sized according to the size of console PCB 336. Second sleeve 324 of flange 316 is designed to allow lower housing 326 to fit therein. Flange 316 performs two functions: (1) to provide mechanical support for holding console PCB 336 and lower housing 326 and (2) to provide electrical isolation between camera PCB 310 and the front metal panel of video console 138, which allows a “floating ground” between removable camera 112 and video console 138.

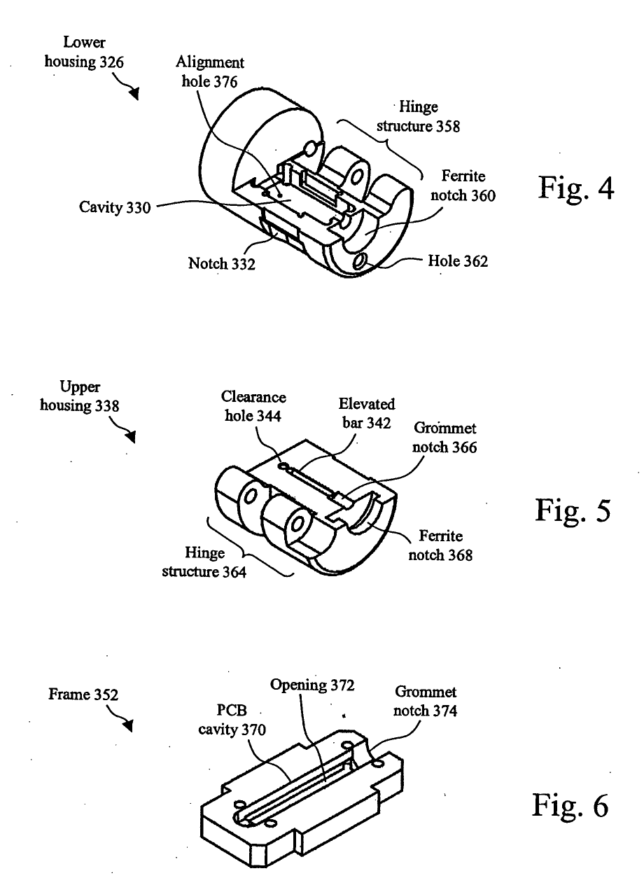

Lower housing 326 is formed of, for example, aluminum or molded plastic. In the case of plastic, ferrite material may optionally be blended within the plastic material to provide an electromagnetic interference (EMI) shield, thereby providing EMI protection. Lower housing 326 is sized to fit into second sleeve 324 of flange 316. A second end of console PCB 336 passes through slot 328, thereby fitting into cavity 330 of lower housing 326. Consequently, slot 328 and cavity 330 are sized according to the size of console PCB 336. Furthermore, cavity 330 is sized and designed to accept frame 352 therein. Lower housing 326 is affixed to flange 316 via screws 356, which are screwed into threaded holes 334. Lower housing 326 is described in more detail in reference to FIG. 4.

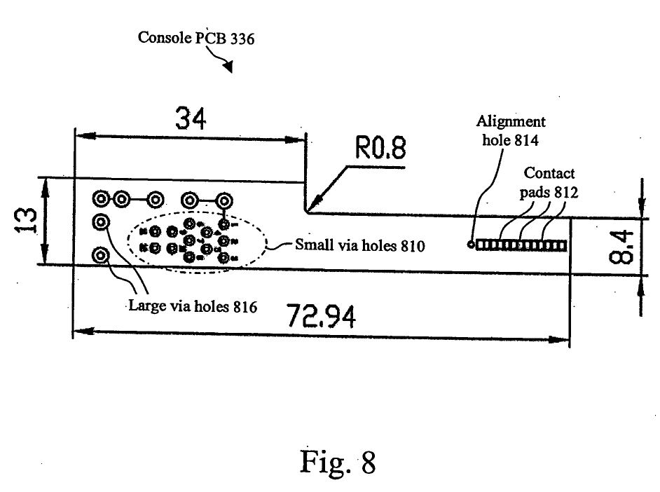

Interconnect 350 is an interconnect system used for board-to-board connections, for example, a GB-type of interconnect system manufactured by Shin-Etsu Polymer Co., Ltd. (Tokyo, Japan) that consists of clusters of conductive regions at predetermined spacing formed of gold-plated brass wires imbedded within silicone rubber, thereby forming a matrix of conductors. Example dimensions of interconnect 350 are 13.7 mm long×3.9 mm wide×1.4 mm thick, that have a conductor pitch of 0.05+0.03/−0.01 mm. Sandwiching interconnect 350 between the contact pads of camera PCB 310 (see FIG. 7) and console PCB 336 (see FIG. 8) and then providing compression realizes a stable, low resistance connection, for example, maximum current per connection is 50 ma and resistance less than 0.1 ohms. The pitch of the conductors within the matrix of interconnect 350 matches the pitch of the contact pads of camera PCB 310 and console PCB 336.

Frame 352 can be formed of molded plastic or any non-conductive material. Camera PCB 310 fits into a recessed opening within frame 352. Once positioned within frame 352, electrical contact pads on the underside of camera PCB 310 are exposed for physical contact to interconnect 350, thereby making an electrical connection between camera PCB 310 and interconnect 350. Frame 352 is described in more detail in reference to FIG. 6.

Console PCB 336 can be a standard multilayer PCB. Console PCB 336 provides the electrical interface between the internal electronics of video console 138 and camera PCB 310. Console PCB 336 is inserted through flange 316, such that a first end is soldered inside video console 138 and the other end protrudes from video console 138 and is then inserted into lower housing 326 for connecting to camera PCB 310. Console PCB 336 is described in more detail in reference to FIG. 8.

Upper housing 338 is formed of, for example, aluminum. Upper housing 338 is hingeably connected to lower housing 326 via hinge screw 340, which serves as the pivot point. When closed, spring latch 346, which is attached to upper housing 338 via screw 348, clamps and locks into notch 332 of lower housing 326, thereby securing upper housing 338 to lower housing 326. Upper housing 338 is described in more detail in reference to FIG. 5.

Elevated bar 342 is an elongated region that is elevated from the surface of upper housing 338, thereby forming a protrusion or bump for applying pressure onto camera PCB 310 and, thus, onto the combination of camera PCB 310, interconnect 350, and console PCB 336 when they are sandwiched together. Example dimensions of elevated bar 342 are 10.7 mm long×1.6 mm wide×1.6 mm high.

Alignment pin 354, which is formed of stainless steel 302, is permanently installed at one end through a hole in console PCB 336 and into lower housing 326. Alignment pin 354 is, for example, 8.5 mm in length and 0.5 mm in diameter. Frame 352 and camera PCB 310 are anchored upon the opposing end of alignment pin 354, which thereby provides an alignment mechanism for the combination of camera PCB 310, frame 352 interconnect 350, and console PCB 336. Clearance hole 344 within upper housing 338 is for accommodating alignment pin 354, when connector assembly 140 is closed.

Ferrite assembly 136, which is described in more detail in reference to FIGS. 10A and 10B, includes ferrite core material, such as manganese and zinc (MnZn) or nickel and zinc (NiZn), for providing EMI protection to removable camera 112, by passing camera cable 120 therethrough. Ferrite assembly 136 is aligned to connector assembly 140 by alignment pin 314, which is inserted into lower housing 326. Furthermore, notches within lower housing 326 and upper housing 338 provide a locking mechanism for ferrite assembly 136, when installed. In an alternative embodiment, ferrite core material may be permanently affixed to lower housing 326 and upper housing 338 of connector assembly 140, instead of having a separate ferrite assembly 136.

FIG. 4 illustrates a perspective view of lower housing 326 in accordance with the invention. In this view, additional details of lower housing 326 are shown. Lower housing 326 further includes a hinge structure 358 for coupling to a complimentary a hinge structure 364 (see FIG. 5) of upper housing 338 via hinge screw 340, a ferrite notch 360, within which ferrite assembly 136 is locked, a hole 362, within which alignment pin 314 of ferrite assembly 136 is inserted, and an alignment hole 376, within which alignment pin 354 (see FIG. 3) of connector assembly 140 is press-fitted.

FIG. 5 illustrates a perspective view of upper housing 338 in accordance with the invention. In this view, additional details of upper housing 338 are shown. Upper housing 338 further includes hinge structure 364 for coupling to the complimentary hinge structure 358 (see FIG. 4) of lower housing 326 via hinge screw 340, a grommet notch 366, within which grommet 312 rests, and a ferrite notch 368, within which ferrite assembly 136 is locked.

FIG. 6 illustrates a perspective view of frame 352 in accordance with the invention. In this view, additional details of frame 352 are shown. Frame 352 further includes a PCB cavity 370, which further includes an opening 372, and a grommet notch 374, within which grommet 312 rests. Camera PCB 310 is installed within PCB cavity 370, such that its contact pads are exposed toward interconnect 350 via opening 372. Additionally, a hole (not visible) with an example diameter of 0.7 mm at the end of PCB cavity 370 that is opposite grommet notch 374 is provided for accepting alignment pin 354 (see FIG. 3). Example overall dimensions of frame 352 are 19.5 mm long×10.4 mm wide×2.4 mm thick.

FIG. 7 illustrates a top view of camera PCB 310 in accordance with the invention. Camera PCB 310 is a standard multilayer PCB formed of any well-known material, such as G10 or FR4, which are created from electrical alkali-free glass cloth that has been impregnated with an epoxy resin. As an example, camera PCB 310 is an eight-layer PCB that has a combination of signal, ground, and power layers, and that has an overall thickness of approximately 1.0 mm. Camera PCB 310 is designed by using standard design rules for ensuring signal integrity, noise immunity, and proper power distribution.

FIG. 7 illustrates example dimensions (in millimeters) of camera PCB 310 and also shows that camera PCB 310 further includes a plurality of solder pads 710, a plurality of contact pads 712, and an alignment hole 714. Alignment hole 714 has an example diameter of 0.7 mm for accepting alignment pin 354 of connector assembly 140 (see FIG. 3).

Solder pads 710 are provided for electrically connecting (via soldering) the conductors within camera cable 120 that supply signals and power to/from removable camera 112. Solder pads 710 and contact pads 712 are electrically connected together by wire traces within the multiple layers of camera PCB 310. Contact pads 712 are provided for contacting the conductors of interconnect 350 when camera PCB 310, interconnect 350, and console PCB 336 are compressed together within connector assembly 140.

The footprint of solder pads 710 and contact pads 712 are identical on each side of camera PCB 310, thereby allowing camera PCB 310 to be installed in connector assembly 140 without regard to orientation, thus making it easy to install by the user. In the specific example shown in FIG. 7, each side of camera PCB 310 includes six solder pads 710 and twelve contact pads 712. Each of the twelve contact pads 712 on one side of camera PCB 310 is electrically connected to its respective opposing pad on the opposite side of camera PCB 310, thereby creating twelve opposing pairs of electrically shorted contact pads 712. By contrast, the six solder pads 710 on one side of camera PCB 310 are electrically isolated from the six solder pads 710 on the opposite side of camera PCB 310. Each of the total of twelve solder pads 710 are electrically connected to one of twelve corresponding pairs of contact pads 712, respectively.

Example dimensions of solder pads 710 are 1.0 mm long; 0.35, 0.45, or 0.55 mm wide; and placed on a pad pitch of 1.0 mm. Likewise, example dimensions of contact pads 712 are 1.3 mm long 0.8 mm wide, placed on a pad pitch of 0.2 mm. The footprint and layout of camera PCB 310 is not limited to that shown in FIG. 7. The number of solder pads 710 and contact pads 712 may be easily expanded, as is practical, while still maintaining the overall dimensions of camera PCB 310.

FIG. 8 illustrates a top view of console PCB 336 in accordance with the invention. Console PCB 336 is a standard multilayer PCB that is formed of any well-known material, such as G10 or FR4. As an example, console PCB 336 is an eight-layer PCB that has a combination of signal, ground, and power layers, and that has an overall thickness of approximately 1.0 mm. Console PCB 336 is designed by using standard design rules for ensuring signal integrity, noise immunity, and proper power distribution.

FIG. 8 illustrates example dimensions (in millimeters) of console PCB 336 and also shows that console PCB 336 further includes a plurality of small via holes 810, a plurality of contact pads 812, an alignment hole 814, and a plurality of large via holes 816. Alignment hole 814 has an example diameter of 0.7 mm for accepting alignment pin 354 of connector assembly 140 (see FIG. 3).

Small via holes 810 and large via holes 816 are plated through holes provided for electrically connecting (via soldering) to circuitry within video console 138 and which supply signals and power to/from removable camera 112. Each contact pad 812 is electrically connected to a respective small via hole 810 by wire traces within the multiple layers of console PCB 336. Contact pads 812 are provided for contacting the conductors of interconnect 350 when camera PCB 310, interconnect 350, and console PCB 336 are compressed together within connector assembly 140. Large via holes 816 are for soldering decoupling capacitors (not shown).

The footprint of contact pads 812 is required on one side only of console PCB 336, i.e., the side facing interconnect 350, when it is installed within connector assembly 140. In the specific example shown in FIG. 8, one side of console PCB 336 includes twelve contact pads 812 that are sized and positioned to mirror contact pads 712 of camera PCB 310, when both are installed within connector assembly 140. The specific I/O assignment of contact pads 812 of console PCB 336 and contact pads 712 of camera PCB 310 also mirror one another.

An example inside diameter of small via holes 810 is 0.7 mm. Likewise, an example inside diameter of large via holes 816 is 1.0 mm. Example dimensions of contact pads 812 are 1.2 mm long 0.8 mm wide, placed on a pad pitch of 0.2 mm. The footprint and layout of console PCB 336 is not limited to that shown in FIG. 8. The number of small via holes 810, contact pads 812, and large via holes 816 may be easily expanded, as is practical, while still maintaining the overall dimensions of console PCB 336.

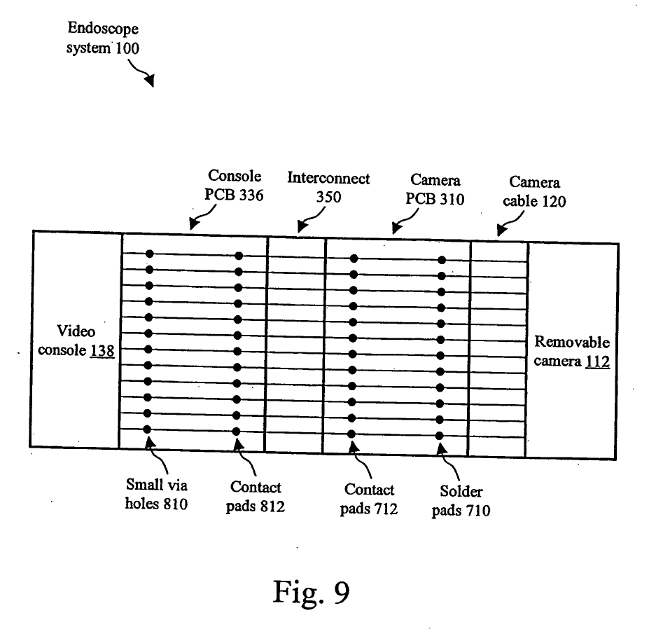

FIG. 9 illustrates an electrical schematic diagram of endoscope system 100 that relates to the signal flow from video console 138 to removable camera 112 in accordance with the invention. FIG. 9 shows electrical connections from video console 138 to small via holes 810 of console PCB 336, then to contact pads 812 of console PCB 336, then through interconnect 350, then to contact pads 712 of camera PCB 310, then to solder pads 710 of camera PCB 310, then through camera cable 120, and then to removable camera 112.

FIGS. 10A and 10B illustrate a cross-sectional view along the length of ferrite assembly 136 and an end view of ferrite assembly 136, respectively, in accordance with the invention. FIGS. 10A and 10B illustrate example dimensions (in millimeters) of ferrite assembly 136 and also show that ferrite assembly 136 further includes a housing 1010, a ferrite core 1012 with a center channel 1014, an endcap 1016, glue epoxy 1018, and alignment pin 314. Alignment pin 314 has an example diameter of 2.5 mm for fitting into hole 362 of lower housing 326 (see FIG. 4) and a length of 25 mm.

Housing 1010 and endcap 1016 are formed of, for example, aluminum. The combination of housing 1010 and endcap 1016 provides a hollow cylinder for holding ferrite core 1012 (which may be installed in multiple segments) along its length. Endcap 1016 is secured to housing 1010 by glue epoxy 1018 after ferrite core 1012 is installed therein. Ferrite core 1012 is formed of, for example, MnZn or NiZn, which is commonly used for forming an EMI shield. Center channel 1014 within ferrite core 1012 provides a passage way for installing camera cable 120 therethrough.

An example outer diameter of housing 1010 is 13 mm and an example inner diameter of housing 1010 is 6.35 mm. An example length of housing 1010 is 70.5 mm. An example outer diameter of ferrite core 1012 is 6.35 mm, and an example inner diameter (thus forming center channel 1014) of ferrite core 1012 is 2.95 mm. The inner diameter of ferrite core 1012 is suitably sized to accept camera cable 120 therethrough.

FIGS. 11 and 12 illustrate example dimensions (in millimeters) of adhesive gasket 319 and adhesive gasket 331, respectively. Adhesive gasket 319 is applied to the video console 138-side of flange 316 and adhesive gasket 331 is applied to the underside of upper housing 338 in order to keep connector assembly 140 splash-proof. Adhesive gasket 319 and adhesive gasket 331 are formed of cutouts from self-adhesive foam, such as Poron 4701-30 manufactured by Rogers Corporation, (Rogers, Conn.). A typical thickness of adhesive gasket 319 and adhesive gasket 331 is in the range of 0.5 to 2.5 mm.

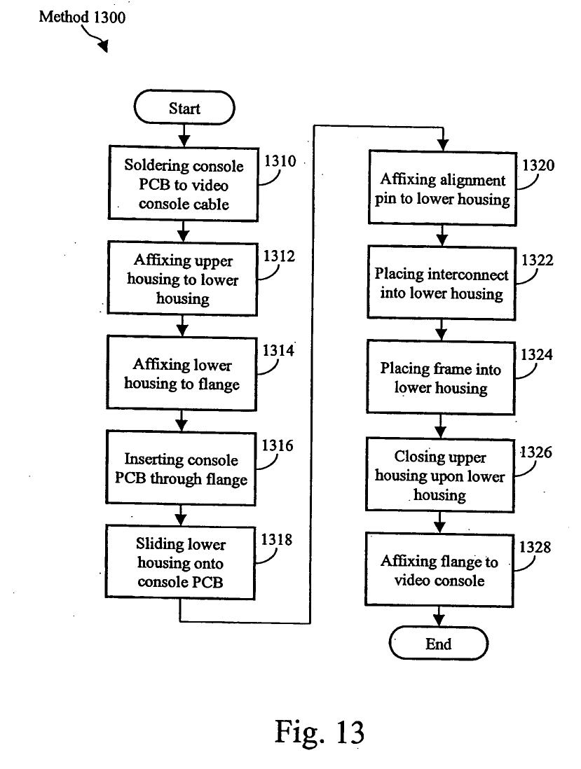

FIG. 13 illustrates a flow diagram of a method 1300 of assembling connector assembly 140 to video console 138 in accordance with one embodiment of the invention. In one embodiment, method 1300 can include the steps of:

Step 1310: Soldering Console PCB to Video Console Cable

In this step, electrical connections are made between a cable within video console 138 and console PCB 336 by soldering electrical wiring of the cable to small via holes 810 and soldering capacitors into large holes 816 of console PCB 336. Method 1300 proceeds to step 1312.

Step 1312: Affixing Upper Housing to Lower Housing

In this step, upper housing 338 is hingeably coupled to lower housing 326 by engaging hinge structure 364 (see FIG. 5) of upper housing 338 with complimentary hinge structure 358 (see FIG. 4) of lower housing 326 via hinge screw 340. Method 1300 proceeds to step 1314.

Step 1314: Affixing Lower Housing to Flange

In this step, lower housing 326 is affixed to flange 316 via screws 356, which are secured into threaded holes 334 of lower housing 326. Method 1300 proceeds to step 1316.

Step 1316: Inserting Console PCB Through Flange

In this step, the end of console PCB 336 that has small via holes 810 and large via holes 816 is inserted through slot 318 of flange 316. Method 1300 proceeds to step 1318.

Step 1318: Sliding Lower Housing onto Console PCB

In this step, slot 328 of lower housing 326 is aligned with and slid over the end of console PCB 336 that has contact pads 812, until the body of lower housing 326 is fitted within second sleeve 324 of flange 316. As a result, the end of console PCB 336 that has contact pads 812 is fitting into cavity 330 of lower housing 326. Method 1300 proceeds to step 1320.

Step 1320: Affixing Alignment Pin to Lower Housing

In this step, alignment pin 354 is permanently installed within lower housing 326 by passing one end of alignment pin 354 through the alignment hole in frame 352, through alignment hole 814 of console PCB 336, and press-fitting into alignment hole 376 of lower housing 326. Method 1300 proceeds to step 1322.

Step 1322: Placing Interconnect into Lower Housing

In this step, interconnect 350 is placed on top of console PCB 336 within cavity 330 of lower housing 326. The conductors of interconnect 350 are automatically aligned with contact pads 812 of console PCB 336. Method 1300 proceeds to step 1324.

Step 1324: Placing Frame into Lower Housing

In this step, frame 352 is placed on top of interconnect 350 within cavity 330 of lower housing 326, such that the conductors of interconnect 350 are aligned with opening 372 of frame 352. Method 1300 proceeds to step 1326.

Step 1326: Closing Upper Housing Upon Lower Housing

In this step, upper housing 338 is closed upon lower housing 326 and secured by engaging spring latch 346, which has been secured to upper housing 338 by screw 348 and notch 332 of lower housing 326. Method 1300 proceeds to step 1328.

Step 1328: Affixing Flange to Video Console

In this step, the top cover of video console 138 is removed and first sleeve 322 of flange 316 is mounted inside video console 138 via a hole in the front panel. Flange 316 is the secured to the front panel of video console 138 via four screws (not shown in FIG. 3), which are installed at mounting holes 320. Method 1300 ends.

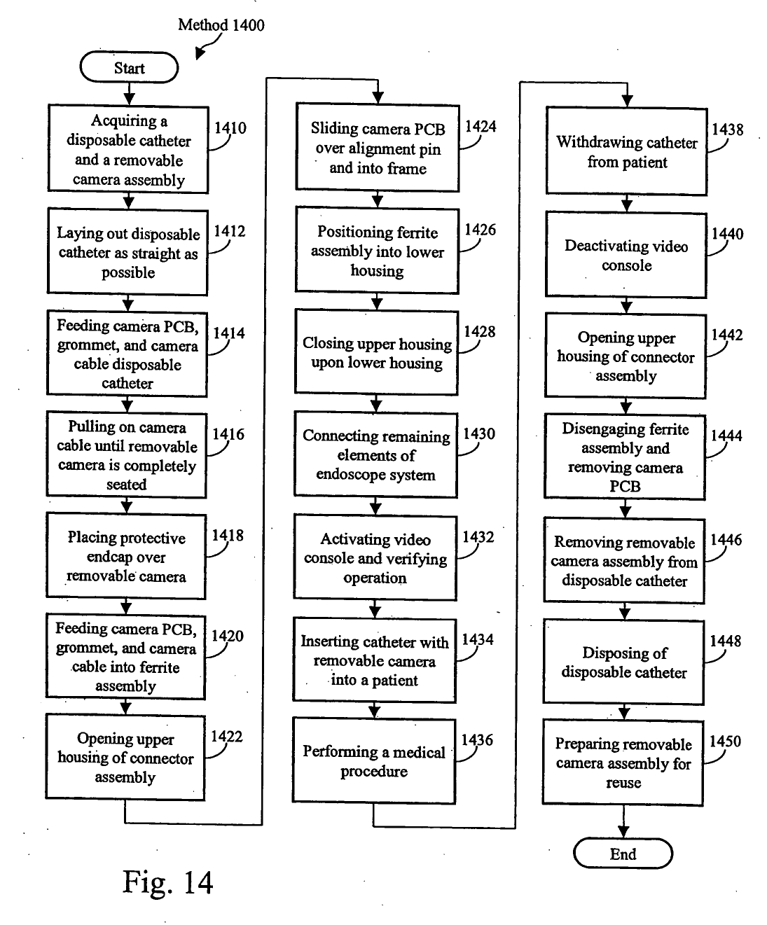

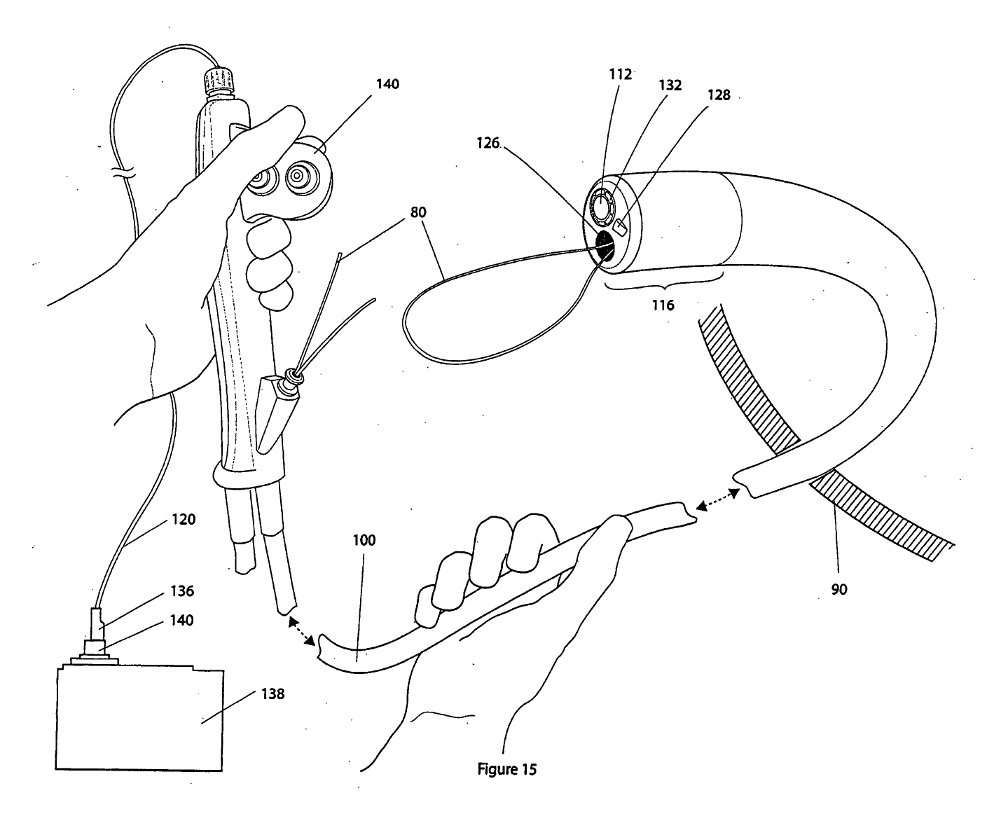

Upon completion of method 1300, flange 316, alignment pin 354, frame 352, interconnect 350, console PCB 336, lower housing 326, and upper housing 338 remain permanently assembled upon video console 138. Only camera PCB 310 is detached/reattached with every use, as described in more detail in FIG. 14.

FIG. 14 illustrates a flow diagram of a method 1400 of using endoscope system 100 in accordance with one embodiment of the invention. In one embodiment, method 1400 includes the steps of:

Step 1410: Acquiring a Disposable Catheter and a Removable Camera Assembly

In this step, a user acquires disposable catheter 110 that is manufactured having, for example, working channel 126, conduit 124, wiring bundle 122, and a channel for accepting camera cable 120. Initially, endcap 118 is hanging separately from disposable catheter 110 by wiring bundle 122, such that the channel for accepting camera cable 120 is accessible. Additionally, the user acquires a removable camera assembly that includes removable camera 112, camera cable 120, camera PCB 310, and grommet 312. Method 1400 proceeds to step 1412.

Step 1412: Laying Out Disposable Catheter as Straight as Possible

In this step, the user lays out, as straight as possible, flexible disposable catheter 110 on a surface. Method 1400 proceeds to step 1414.

Step 1414: Feeding Camera PCB, Grommet, and Camera Cable into Disposable Catheter

In this step, the user feeds camera PCB 310, grommet 312, and camera cable 120 into catheter distal end 116 of disposable catheter 110, until camera PCB 310 emerges from catheter proximal end 114 of disposable catheter 110. Method 1400 proceeds to step 1416.

Step 1416: Pulling on Camera Cable Until Removable Camera is Completely Seated

In this step, the user pulls gently upon camera cable 120, which is extending out of catheter proximal end 114 of disposable catheter 110, until removable camera 112 is seated within catheter distal end 116 of disposable catheter 110. More specifically, by pulling upon camera cable 120, removable camera 112 snaps into a square, recessed hole that has a rubber gasket to hold removable camera 112 in place by pressure. Method 1400 proceeds to step 1418.

Step 1418: Placing Protective Endcap Over Removable Camera

In this step, the user secures endcap 118 upon catheter distal end 116 of disposable catheter 110 with removable camera 112 aligned with illumination assembly 132, as shown in FIG. 2. Method 1400 proceeds to step 1420.

Step 1420: Feeding Camera PCB, Grommet, and Camera Cable into Ferrite Assembly

In this step, the user feeds camera PCB 310, grommet 312, and camera cable 120 into the endcap 1016-end of ferrite assembly 136, until camera PCB 310 emerges from the opposing end of ferrite assembly 136. Method 1400 proceeds to step 1422.

Step 1422: Opening Upper Housing of Connector Assembly

In this step, the user disengages spring latch 346 of upper housing 338 from notch 332 of lower housing 326, thereby allowing upper housing 338 to swing open via its hinge assembly, thus providing accessibility to frame 352 and alignment pin 354. Method 1400 proceeds to step 1424.

Step 1424: Sliding Camera PCB Over Alignment Pin and into Frame

In this step, without regard to the top-side or bottom-side orientation of camera PCB 310, the user aligns alignment hole 714 of camera PCB 310 to alignment pin 354 of connector assembly 140 and, subsequently, slides camera PCB 310 onto alignment pin 354, until camera PCB 310 is seated within PCB cavity 370 of frame 352. As a result, contact pads 712 of camera PCB 310 are aligned with the conductors of interconnect 350. Method 1400 proceeds to step 1426.

Step 1426: Positioning Ferrite Assembly into Lower Housing

In this step, the user slides ferrite assembly 136 along camera cable 120 and engages its alignment pin 314 within hole 362 of lower housing 326, thereby seating ferrite assembly 136 into ferrite notch 360 of lower housing 326. Method 1400 proceeds to step 1428.

Step 1428: Closing Upper Housing Upon Lower Housing

In this step, the user closes upper housing 338 upon lower housing 326 by engaging spring latch 346 with notch 332 of lower housing 326. In doing so, elevated bar 342 applies pressure to camera PCB 310, which subsequently applies pressure to the aligned combination of camera PCB 310, interconnect 350, and console PCB 336, thereby achieving a reliable, low-resistance connection therebetween. Additionally, by closing upper housing 338, ferrite assembly 136 is engaged and locked into ferrite notch 368 of upper housing 338. Method 1400 proceeds to step 1430.

Step 1430: Connecting Remaining Elements of Endoscope System

In this step, the user couples any remaining elements of endoscope system 100. For example, conduit 124 and wiring bundle 122 are coupled to fluid supply 130 and illumination controller 134, respectively; thus, endoscope system 100 is ready for use. Method 1400 proceeds to step 1432.

Step 1432: Activating Video Console and Verifying Operation

In this step, the user activates video console 138 and verifies that removable camera 112 is properly connected and operational, by observing images received from removable camera 112 upon the video display of video console 138. Method 1400 proceeds to step 1434.

Step 1434: Inserting Catheter with Removable Camera into a Patient

In this step, a physician inserts catheter distal end 116 of disposable catheter 110 into a body cavity of a patient. Method 1400 proceeds to step 1436.

Step 1436: Performing a Medical Procedure

In this step, a physician performs the inspection procedure by manipulating fluid supply 130, illumination controller 134, video console 138, and motion controller 142, as needed. Method 1400 proceeds to step 1438.

Step 1438: Withdrawing Catheter from Patient

In this step, a physician withdraws catheter distal end 116 of disposable catheter 110 from the body cavity of the patient. Method 1400 proceeds to step 1440.

Step 1440: Deactivating Video Console

In this step, having printed the images or storing in memory any desired images captured in step 1438, the user deactivates video console 138. Method 1400 proceeds to step 1442.

Step 1442: Opening Upper Housing of Connector Assembly

In this step, the user disengages spring latch 346 of upper housing 338 from notch 332 of lower housing 326, thereby allowing upper housing 338 to swing open via its hinge assembly and, thus, providing accessibility to camera PCB 310. Method 1400 proceeds to step 1444.

Step 1444: Disengaging Ferrite Assembly and Removing Camera PCB

In this step, the user disengages ferrite assembly 136 from lower housing 326 by sliding ferrite assembly 136 away from lower housing 326 along camera cable 120. The user then slides camera PCB 310 off alignment pin 354, thereby removing camera PCB 310 from PCB cavity 370 of frame 352 and freeing the removable camera assembly from connector assembly 140. Method 1400 proceeds to step 1446.

Step 1446: Removing Removable Camera Assembly from Disposable Catheter

In this step, the user disassembles endcap 118 from catheter distal end 116 of disposable catheter 110, thereby exposing removable camera 112. The user then gently pushes upon the proximal end of camera cable 120 to slightly dislodge removable camera 112 from the recessed hole and rubber gasket within which it was installed. The user is then able to grasp removable camera 112 and remove it from disposable catheter 110 by pulling its camera cable 120, grommet 312, and camera PCB 310 out of disposable catheter 110. Method 1400 proceeds to step 1448.

Step 1448: Disposing of Disposable Catheter

In this step, the user disposes of disposable catheter 110 by using proper procedures of disposing medical devices. Method 1400 proceeds to step 1450.

Step 1450: Preparing Removable Camera Assembly for Reuse

In this step, the user prepares the removable camera assembly, which includes removable camera 112, camera cable 120, camera PCB 310, and grommet 312, for reuse by soaking in, for example, a standard cleaning substance, such as a Cidex Solution supplied by Johnson & Johnson (New Brunswick, N.J.), for a predetermined length of time, thereby cleaning and disinfecting the removable camera assembly. Method 1400 ends.

FIG. 15 illustrates a perspective view of an embodiment of endoscope system 100 being used within a body cavity. The catheter distal end 116 is positioned within the body, illustrated by presence of a body wall 90. The distal end 116 can be inserted into a naturally occurring body orifice (eg. mouth, anus), or alternatively, can be inserted into an opening made in the body wall 90. Proximal end 114 of the catheter remains outside the body for manipulation by the user. In this illustration, a looped guidewire 80 is present within working channel 126 for assistance in navigation through the body. The following US patent applications disclose various assemblies for advancing a medical device along a guidewire, and are incorporated herein by reference: U.S. Ser. No. 10/409,270 filed Apr. 8, 2003; U.S. Ser. No. 10/729,754 filed Dec. 5, 2003; U.S. Ser. No. 10/406,020 filed Apr. 3, 2003; Ser. No. 10/310,365 filed Dec. 5, 2002; U.S. Provisional Application 60/571,118 filed May 14, 2004, and U.S. Provision Application 60/571,026 filed May 14, 2004.

Video equipment that remains outside the body, such as video console 138, connector assembly 140, and ferrite assembly 136 are also shown in FIG. 15. Other capital equipment such as illumination controller 134, air supply 131, fluid supply 130, and vacuum source 142 also remain outside the body, but are not shown.

Endoscope system 100 of the present invention provides disposable catheter 110 that is discarded after a single use, a reusable camera by way of removable camera 112 that is cleaned after each use, and an improved camera connector system by way of connector assembly 140 for quickly and easily coupling to video console 138. More specifically, the reuse of removable camera 112 is made possible by camera PCB 310, which is suitably small enough to be fed into a very narrow channel (i.e., less than 2.8 mm diameter) within disposable catheter 110 for easy installation in its distal end. The small size of camera PCB 310 allows the overall diameter of disposable catheter 110 to be maintained as small as possible. Connector assembly 140 provides a simple and efficient means for quickly and easily attaching/detaching camera PCB 310 of removable camera 112 to video console 138.

As a result, endoscope system 100 of the present invention provides a cost-effective method of increasing a physician's daily throughput in relation to the number of endoscope inspection procedures performed, by allowing several removable cameras 112 and disposable catheters 110 to be purchased, thus available, for use. In this way, instead of cycling a limited number of reusable endoscope systems through a time-consuming cleaning process each day, as is customary, a greater number of less expensive removable cameras 112, which are used in combination with disposable catheters 110, are on hand and cycled through the cleaning process with each use, thereby increasing a physician's daily throughput in relation to the number of endoscope inspection procedures performed.

While the present invention has been illustrated by description of various embodiments, it is not the intention of the applicants to restrict or limit the spirit and scope of the appended claims to such detail. Numerous other variations, changes, and substitutions will occur to those skilled in the art without departing from the scope of the invention. Moreover, the structure of each element associated with the present invention can be alternatively described as a means for providing the function performed by the element. It will be understood that the foregoing description is provided by way of example, and that other modifications may occur to those skilled in the art without departing from the scope and spirit of the appended claims.

1. A medical device comprising:
an elongate flexible member adapted for extending from a point outside a patient's body to a point inside the patient's body, and having at least one passageway for communicating from a point outside the body to a point inside the body, the passageway having a distal end and a proximal end;
a camera adapted to be associated with a distal portion of the elongated flexible member for receiving images within the patient's body; and
an assembly operatively associated with the camera and sized to be receivable in the distal end of the passageway and to pass through the proximal end of the passageway in the elongated flexible member, the assembly being positioned outside of the patient's body during use of the medical device.

2. The medical device of claim 1 wherein the assembly comprises electrical circuitry.

3. The medical device of claim 1 wherein the assembly comprises a printed circuit board.

4. The medical device of claim 1 wherein the assembly comprises a camera printed circuit board.

5. The medical device of claim 1 wherein the assembly has a maximum width less than about 3.0 mm.

6. The medical device of claim 5 wherein the assembly has a maximum width which is less than about 2.8 mm.

6. The medical device of claim 1 wherein the assembly comprises a plurality of contact pads.

7. The medical device of claim 1 wherein the assembly comprises a plurality of solder pads.

8. The medical device of claim 1 wherein the assembly comprises a printed circuit board having a plurality of solder pads and contact pads on each side thereof, and wherein the footprint of the solder pads and contact pads are identical on each side of the printed circuit board.

9. The medical device of claim 1 comprising a camera cable extending through the passageway from the camera to the assembly.

10. The medical device of claim 9 wherein the assembly comprises a housing, a core disposed within the housing, and a channel within the core for receiving a proximal portion of the camera cable therethrough.

11. The medical device of claim 1 further comprising a connector assembly disposed outside the patient's body, wherein the connector assembly is adapted to releasably engage the assembly operatively associated with the camera, and wherein the connector assembly is operatively associated with a video unit.

12. The medical device of claim 11 wherein the assembly operatively associated with the camera comprises a first printed circuit board, and wherein the connector assembly comprises a second printed circuit board.

13. The medical device of claim 11 wherein the first printed circuit board is disposed within the connector assembly when the connector assembly releasably engages the assembly operatively associated with the camera.

14. The medical device of claim 12 wherein the connector assembly further comprises an interconnect for providing board to board contact between the first and second printed circuit boards.

15. An apparatus for use with an endoscopic device, the apparatus comprising:
a camera sized and adapted to be disposed on a distal end of the endoscopic device;
a camera cable having a diameter sized to be received through the endoscopic device, the cable extending from the camera and having a length sufficient to extend from the camera when the camera is inside a patient's body to a position outside of a patient's body; and
a camera cable end assembly disposed on a proximal end portion of a camera cable, the cable end assembly comprising electrical circuitry operatively associated with the camera through the cable.

16. The apparatus of claim 15 wherein the cable end assembly comprises a printed circuit board.

17. The apparatus of claim 15 wherein the cable end assembly has a maximum width of less than about 3.0 mm.

18. The apparatus of claim 17 wherein the cable end assembly has a maximum width of less than about 2.8 mm.

19. The apparatus of claim 15 wherein the cable end assembly comprises a plurality of solder pads.

20. The apparatus of claim 15 wherein the cable end assembly comprises a plurality of contact pads.

21. The apparatus of claim 15 further comprising a connector assembly for releasably engaging the camera cable end assembly.

22. The apparatus of claim 21 wherein the cable end assembly comprises a first printed circuit board, and wherein the connector assembly is adapted to releasably engage the cable end assembly printed circuit board.

23. The apparatus of claim 22 wherein the connector assembly comprises a frame for receiving the cable end assembly printed circuit board.

24. The apparatus of claim 21 wherein the connector assembly comprises a second printed circuit board.

25. The apparatus of claim 24 wherein the connector assembly further comprises an interconnect for providing board to board connection of the first printed circuit board with the second printed circuit board.

26. The apparatus of claim 21 wherein the connector assembly comprises housing having a first housing portion and a second housing portion, the first housing portion pivotable with respect to the second housing portion to open the housing, and wherein at least a portion of the cable end assembly is adapted to be releasably received within the housing.
